(19) United States
(12) Patent Application Publication
(10) Pub. No.: US 2005/0182458 A1
(43) Pub. Date: Aug. 18, 2005

(54) Implantable medical devices with dual-memory support

(75) Inventor: Steven D. Goedeke, Forest Lake, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/781,463

(22) Filed: Feb. 17, 2004

(51) Int. Cl. A61N 1/08

(52) U.S. Cl. 607/59

(57) ABSTRACT

The invention is directed to implantable medical devices (IMDS) with dual-memory support. An IMD is designed to detect the presence or absence of a programmable non-volatile memory, such as flash memory. The IMD processor determines whether operation instructions reside in non-programmable non-volatile memory or in programmable non-volatile memory as a function of an output from a detector circuit.

The invention relates to medical devices and, more particularly, to implantable medical devices.

### BACKGROUND

An implantable medical device (IMD) typically performs therapeutic functions in response to detected physiologic or received control signals. The therapeutic functions performed by the IMD vary from device to device. A cardiac pacemaker, for example, monitors heart rate and rhythm, and applies stimulation therapy when specific arrhythmic conditions are encountered. An implanted drug-delivery device may monitor any number of physiological factors and administer medications as appropriate. Other examples of IMDs include physiologic monitors, nerve stimulators, muscle stimulators, brain stimulators, cochlear implants, implantable defibrillators, and the like.

Each IMD generally includes a processor that executes “operation instructions” or applies “operation code” to carry out the various operational functions of the IMD. Typical operation instructions are stored in one or more non-volatile memory modules in the IMD. Non-volatile memory includes, for example, conventional read-only memory (ROM).

Each IMD has a “manufacturing life cycle,” which represents a time period over which various models of the same product are made. The software supplied with an IMD early in the manufacturing life cycle may be different from the software supplied with the IMD later in the manufacturing life cycle. Clinical experience, production efficiency, modifications and improvements may impose a need to change the operation instructions.

### SUMMARY

In general, the invention is directed to implantable medical devices (IMDs) with two types of memory support. Specifically, an IMD is designed to support at least two kinds of non-volatile memory having programmable and non-programmable features. Each kind of non-volatile memory can hold instructions for device operations.

In the early part of the manufacturing life cycle, it is often desirable for an IMD to have flexibility and versatility in its firmware. Specifically, flexibility around features such as therapeutic or diagnostic functions is desirable during the early manufacturing life cycle so that the physician and the IMD manufacturer may assess the performance of the IMD and make changes as needed. It is also not uncommon to add, delete, modify or adjust operation instructions in the early stages of a new product. Such modifications may be made when the IMD processor loads operation instructions from the programmable non-volatile memory. The frequency of such changes diminishes as the IMD's manufacturing life cycle matures.

In an embodiment of the invention, an IMD includes a detector circuit to determine whether programmable non-volatile memory is present. The IMD processor determines whether operation instructions reside in non-programmable non-volatile memory or in programmable non-volatile memory as a function of an output from the detector circuit. The processor may make this determination after a power-up or a reset, for example.

The details of one or more embodiments of the invention are set forth in the accompanying drawings and the description below.

### BRIEF DESCRIPTION OF DRAWINGS

FIG. 1 is a block diagram illustrating a first model of an implantable medical device (IMD) with both non-programmable non-volatile memory and programmable non-volatile memory.

FIG. 2 is a block diagram illustrating a second model of the IMD from FIG. 1, with non-programmable non-volatile memory.

### DETAILED DESCRIPTION

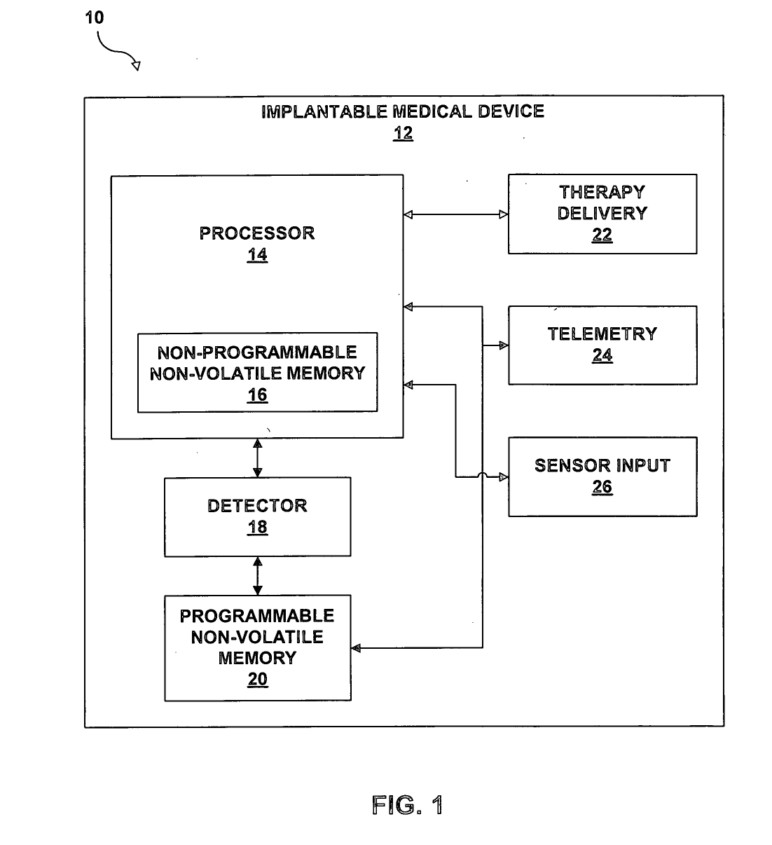

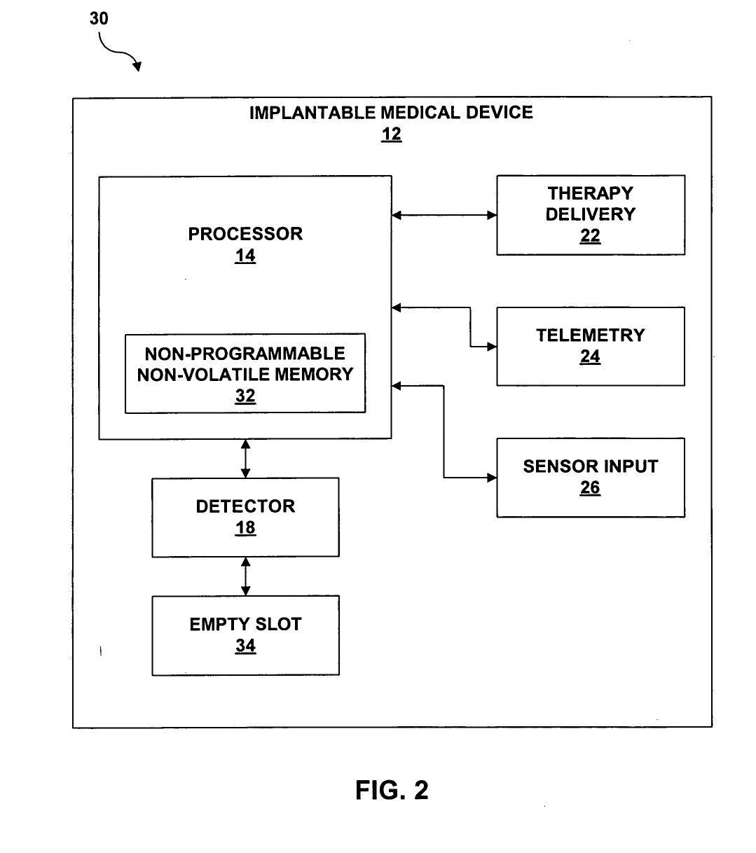

FIGS. 1 and 2 are block diagrams illustrating two models 10, 30 of an implantable medical device (IMD) 12. As used herein, the “models” are not different products, but are different versions of the same IMD. Indeed, models 10, 30 are alike in many respects, but differ in storage of operation instructions. IMD 12 includes flexibility to retrieve operation instructions from a non-programmable non-volatile memory module 16 or a programmable non-volatile memory module 20. Model 10 includes programmable non-volatile memory 20, and IMD processor 14 loads operation instructions from programmable non-volatile memory 20. With model 30, by contrast, IMD processor 14 loads operation instructions from non-programmable non-volatile memory 32.

IMD 12 comprises without limitation one or more of a variety of implantable devices, including a cardiac pacemaker, a physiologic monitor, a drug dispenser, a nerve stimulator, a muscle stimulator, a brain stimulator, a cochlear implant, a blood pump, a cardiomyostimulator, a tachyarrhythmia-control device, and an implantable defibrillator. The invention is not limited to the particular devices listed. For purposes of illustration, the invention may be described in the context of IMD 12 being an implantable pacemaker-defibrillator.

In one embodiment, IMD 12 receives physiologic signals from at least one sensor 26 and delivers therapy to a patient via a therapy delivery module 22. Sensor 26 includes sensors that detect any quantity, such as pressure, electrical activity, impedance, temperature, blood chemistry, analyte concentration, and the like. Therapy delivery module 22 includes any therapy delivery device, such as an electrode to deliver stimulation or a drug delivery apparatus.

As depicted in FIG. 1, first model 10 of IMD 12 includes a processor 14 with an embedded non-programmable non-volatile memory 16, such as conventional ROM. Although non-programmable non-volatile memory 16 is depicted as an element of processor 14, the invention also includes embodiments in which non-programmable non-volatile memory 16 is distinct from processor 14. Processor 14 can be embodied as a microprocessor, a controller, a digital signal processor, an application specific integrated circuit, a field-programmable gate array, discrete logic circuitry, or the like.

First model 10 also includes programmable non-volatile memory 20, and a detector circuit 18, which detects the presence or absence of programmable non-volatile memory 20. Detector circuit 18 comprises any circuit that can detect the presence of programmable non-volatile memory 20. In one embodiment, detector circuit 18 comprises a transistor that generates a “high” or “low” voltage output depending upon whether programmable non-volatile memory 20 is present to provide a current path. The “high” or “low” voltage output maps to a logical value that signifies whether programmable non-volatile memory 20 is present or absent.

When detector circuit 18 generates a signal that indicates the presence of programmable non-volatile memory 20, processor 14 receives and processes the signal from the detector circuit 18. When the presence of programmable non-volatile memory 20 is confirmed, processor 14 loads operation instructions stored in programmable non-volatile memory 20 and executes the operation instructions accordingly.

In one embodiment, a reset or power-up operation may trigger processor 14 to check for a signal from detector circuit 18, to load one or more operation instructions from the programmable non-volatile memory when said presence is confirmed, and to execute the appropriate subsequent instruction. However, since power-ups and resets are not frequently encountered in the operation of IMD 12, processor 14 may not be routinely engage in this operation.

First model 10 represents IMD 12 in the early stages of the manufacturing life cycle of IMD 12. First model 10 stores operation instructions for IMD 12 in programmable non-volatile memory 20. First model 10 may also store operation instructions in non-programmable non-volatile memory 16, but instructions in non-programmable non-volatile memory 16 will generally not be accessed. More specifically, in some embodiments, the programmable memory 20 will be used exclusively if present, while in other embodiments some data may be accessed from the non-programmable memory 16 even when the programmable memory 20 is present. When, processor 14, determines that programmable non-volatile memory 16 is present based upon a signal from detector 18, the processor 14 loads operation instructions from programmable non-volatile memory 20.

First model 10 may be implanted in the body of a patient. In a typical manufacturing scenario, a manufacturer produces many first model IMDs 10 that are implanted in patients. Each first model IMD 10 includes non-programmable non-volatile memory 16 and programmable non-volatile memory 20.

There are many reasons for modification of operation instructions early in the manufacturing life cycle. For example, physicians may wish to enable features such as therapeutic or diagnostic functions, so that both the physicians and the IMD manufacturer may assess the performance of IMD 12. In addition, the manufacturer may issue updates to the operation instructions, which can be written to programmable non-volatile memory 20.

In the embodiments depicted in FIGS. 1 and 2, IMD 12 includes a telemetry module 24. A physician, clinician or IMD manufacturer changes operation instructions by transmitting programming from an external programmer (not shown) via telemetry module 24. Telemetry module 24 may include any wireless system for transmitting and receiving between IMD 12 and an external programmer. A typical telemetry module telemeters radio frequency (RF) encoded signals. An external programmer changes operation instructions stored in programmable non-volatile memory 20, and can also direct processor 14 to utilize the newly programmed operation instructions.

Because operation instructions stored in programmable non-volatile memory 20 can be modified, IMD 12 is versatile in operation. Different functionalities may be enabled, disabled or otherwise changed, and the physician and the manufacturer may assess the performance of IMD 12 under a variety of operating conditions. In this way, the physician and the manufacturer could enhance the utility or functionality of IMD 12.

After a period of time, however, operating instructions usually stabilize. Specifically, a standard set of instructions will be established and operating instructions mature for a given model of IMD 12. The stabilization period varies from device to device, and also depends upon the number of patients that are implanted with an IMD of that particular model. The operating instructions for a typical implantable device can stabilize in about ninety days to three years.

A cardiac pacemaker, for example, early in its manufacturing life cycle may include several routines for detection of heart rhythms, and for classifying the rhythms. Each of these routines can be embodied in operation instructions that are stored in programmable non-volatile memory. The routines may be enabled or disabled or modified in several patients, and the efficacy of the routines may be judged. After a stabilization period, such as a year, the operating instructions for the pacemaker stabilize. Thus, certain therapeutic or diagnostic functions may be enabled or disabled on a full-time basis. Updates to the operation instructions become unnecessary.

Once the operation instructions have stabilized, it is undesirable to include programmable non-volatile memory in IMD 12, because such programmable non-volatile memory would increase the cost of the device without providing significant benefit.

Accordingly, manufacturer issues a second model 30 of IMD 12. Second model 30 may be very similar to first model 10. In some implementations, second model 30 may be identical to first model 10 in all aspects except for the absence of non-volatile memory. Second model 30 includes a connector element such as empty slot 34 that is configured to couple to a programmable non-volatile memory module, but that couples to no such module. When triggered by a reset or power-up, for example, detector 18 generates a signal that indicates the absence of programmable non-volatile memory. Processor 14 confirms the signal and loads operation instructions from non-programmable non-volatile memory 32. Non-programmable non-volatile memory 32, which may be different from non-programmable non-volatile memory 16 in first model 10, stores at least one operation instruction identical to an operation instruction stored in programmable non-volatile memory 20 of first model 10.

Thus, once standardized operation instructions exist, the manufacturer can eliminate the extraneous memory costs from the manufacturing process without having to redesign or modify the device model or the assembly process. The redesign of a medical device is a costly process and requires re-evaluation of the safety and efficiency of the new product as well as extensive and burdensome modifications to the assembly process.

The invention is not limited to applications in which operation instructions load directly from non-programmable non-volatile memory or programmable non-volatile memory into a processor. The invention encompasses embodiments in which the operation instructions are stored in an intermediate memory element, such as a memory cache. The invention also encompasses embodiments in which different models of an IMD are used for different purposes. These and other embodiments are within the scope of the following claims.

1. A method comprising:
providing a first model of an implantable medical device, the first model of the implantable medical device including a non-programmable non-volatile memory and a programmable non-volatile memory; and
providing a subsequent model derived from the first model, a second model of the implantable medical device, wherein the subsequent model including non-programmable non-volatile memory and excluding the programmable non-volatile memory.

2. The method of claim 1, wherein the programmable non-volatile memory in the first model stores operation instructions.

3. The method of claim 1, wherein the programmable non-volatile memory comprises one of a flash memory, an electrically erasable programmable read-only memory (EEPROM), and a non-volatile random-access memory (NVRAM).

4. The method of claim 1, wherein the non-programmable non-volatile memory comprises a read-only memory (ROM).

5. The method of claim 1, further comprising waiting for a stabilization period after providing the first model and before providing the second model.

6. The method of claim 4, wherein the stabilization period comprises ninety days to one year.

7. The method of claim 1, further comprising manufacturing the first and second models of the implantable medical device including a detector circuit configured to detect one of a presence and an absence of the programmable non-volatile memory.

8. The method of claim 1, wherein the non-programmable non-volatile memory in the second model stores at least one operation instruction stored in the programmable non-volatile memory of the first model.

9. A method comprising:
confirming one of a presence and an absence of a programmable non-volatile memory in an implantable medical device;
loading an operation instruction from the programmable non-volatile memory when said presence is confirmed; and
loading an operation instruction from a non-programmable non-volatile memory when said absence is confirmed.

10. The method of claim 8, wherein confirming one of a presence and an absence of a programmable non-volatile memory comprises first detecting a condition of the implantable medical device.

11. The method of claim 8, wherein the implantable medical device comprises at least one of a cardiac pacemaker, a physiologic monitor, a drug dispenser, a nerve stimulator, a muscle stimulator, a brain stimulator, a cochlear implant, a blood pump, a cardiomyostimulator, and a tachyarrhythmia-control device.

12. An implantable medical device comprising:
a processor;
a non-programmable, non-volatile memory circuit.
a connector element configured to couple to a programmable non-volatile memory module; and
a detector circuit in operable electrical contact with the connector element configured to detect the presence of the programmable non-volatile memory, wherein the detector circuit is in communication with the processor, and the processor determines where to obtain operating instructions, based on an output from the detector circuit.

13. The implantable medical device of claim 11, further comprising a non-programmable non-volatile memory module.

14. The implantable medical device of claim 11, wherein the implantable medical device comprises one of a cardiac pacemaker, a physiologic monitor, a drug dispenser, a nerve stimulator, a muscle stimulator, a brain stimulator, a cochlear implant, a blood pump, a cardiomyostimulator, and a tachyarrhythmia-control device.

15. The implantable medical device of claim 11, wherein the programmable non-volatile memory module comprises one of a flash memory, an electrically erasable programmable read-only memory (EEPROM), and a non-volatile random-access memory (NVRAM).
